(19) United States
(12) Patent Application Publication
(10) Pub. No.: US 2007/0237307 A1
(43) Pub. Date: Oct. 11, 2007

(54) Radiographic spine marker

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/368,052

(22) Filed: Mar. 3, 2006

(51) Int. Cl. A61B 6/08

(52) U.S. Cl. 378/205

(57) ABSTRACT

A radiographic spine marker includes a flexible, radiolucent platform having notches along its edges, and perforations to provide a marking template, and radiopaque markings associated with the notches and perforations, to help the surgeon identify the location of the notches during radiographic visualization.

### BACKGROUND OF THE INVENTION

This invention relates to orthopedic surgery, and more particularly to a radiographic spine marker for facilitating more precise placement of surgical spinal prostheses.

### BACKGROUND OF THE INVENTION

Radiographic markers may be applied to the surface of a vertebral body or intervertebral disc to facilitate the accurate placement of spinal surgical prostheses used in both fusion and nonfusion spinal surgery.

Of all animals possessing a backbone, human beings are the only creatures who remain upright for significant periods of time. From an evolutionary standpoint, this erect posture has conferred a number of strategic benefits, not the least of which is freeing the upper limbs for purposes other than locomotion. From an anthropologic standpoint, it is also evident that this unique evolutionary adaptation is a relatively recent change, and as such has not benefited from natural selection as much as have the horizontal backbones of other animals. As a result, stresses acting upon the human backbone (or “vertebral column”) are unique in many senses, and result in a variety of problems or disease states that are peculiar to the human species.

The human vertebral column is essentially a tower of bones held upright by fibrous bands called ligaments and contractile elements called muscles. There are seven bones in the neck or cervical region, twelve in the chest or thoracic region, and five in the low back or lumbar region. There are also five bones in the pelvic or sacral region which are normally fused together and form the back part of the pelvis. This column of bones is critical for protecting the delicate spinal cord and nerves, and for providing structural support for the entire body.

Between the vertebral bones themselves exist soft tissue structures—discs—composed of fibrous tissue and cartilage which are compressible and act as shock absorbers for sudden downward forces on the upright column. The discs allow the bones to move independently of each other, as well. The repetitive forces which act on these intervertebral discs during repetitive day-to-day activities of bending, lifting and twisting cause them to break down or degenerate over time.

Presumably because of humans' upright posture, their intervertebral discs have a high propensity to degenerate. Overt trauma, or covert trauma occurring in the course of repetitive activities disproportionately affect the more highly mobile areas of the spine. Disruption of a disc's internal architecture leads to bulging, herniation or protrusion of pieces of the disc and eventual disc space collapse. Resulting mechanical and even chemical irritation of surrounding neural elements (spinal cord and nerves) cause pain, attended by varying degrees of disability. In addition, loss of disc space height reduces tension on the longitudinal spine ligaments, thereby contributing to spinal instabilities such as spinal curvature, and lithesis.

Time-honored methods of addressing neural irritation and instability resulting from severe disc damage have largely focused on removal of the damaged disc and fusing the adjacent vertebral elements together. Removal of the disc relieves the mechanical and chemical irritation of neural elements, while osseous union (bone knitting) solves the problem of instability.

More recently, total disc replacement has become an alternative to fusion in regards to addressing issues of disc degeneration and associated degenerative instability. These advances in technology have made it even more imperative that the spinal prosthesis being inserted be exactly positioned in order to function favorably in accordance with the biomechanical forces acting within and upon the normal spine.

In most instances of anterior fusion of the spine, identification of the true center portion of the spine is difficult or impossible with simple direct visualization afforded through the limited exposures favored by minimally invasive techniques. As an adjunct, intra operative x-ray is utilized to assist in finding the true midline of the spine in an effort to insure that bone grafts, interbody fusion devices, or spinal fusion plates are properly centered. Proper centering of these grafts and/or associated spinal hardware maximizes their biomechanical advantage and facilitates faster and better bone knitting and spinal healing.

In the case of total disc replacement, centering of the prosthesis is of even greater importance to insure proper functioning of the device, to prevent excessive mechanical wear, and to prevent dislodgement of the device itself from unanticipated or improper biomechanical forces acting on the device secondary to malposition at the time of surgical implantation.

To address the issue of proper placement of the fusion and nonfusion devices, various metal pins or screws have been placed in the vertebral bodies or discs at the time of surgery and the location of the metallic markers has been documented radiographically at the time of surgery. The markers are then moved or repositioned, and repeat x-rays are obtained until an approximation of the center of the spine has been achieved.

Clearly, there is a need in the art to have a swifter and more accurate means of centering or positioning these spinal prostheses to insure optimal function of the devices and to lessen the surgical time necessary in achieving these ends.

### SUMMARY OF THE INVENTION

An object of the invention is to provide a simple, rapid and accurate means of localizing the anatomic center of a spinal vertebrae or intervertebral disc. It is also an object of this invention to provide for a means of accurately measuring spinal characteristics such as disc or vertebral width without having to account for magnification artifacts attendant with all x-ray imaging. An additional object is to provide for a rapid means of marking central and paracentral landmarks on the vertebral body or intervertebral disc surface to facilitate various spinal surgeries.

To achieve these objects, a curved radiolucent platform is employed, having roughly the surface curvature of the anterior portion of an average spinal vertebrae or intervertebral disc. The platform is flexible, so as to conform to minor variations in the curvature between individual spines. In the preferred embodiment, various plastics, fabrics or synthetics may be employed to formulate the platform, provided they are radiolucent.

Affixed to this curved platform are radiopaque markers that are situated, much like the demarcations on a ruler. These marks are separated in distances of one or more millimeters depending on the accuracy needed for the surgical procedure in question. The marks are of sufficient size so as to be visible on a standard AP, lateral, or fluoroscopic x-ray.

On the concave surface of the platform is one or more pins which may or may not be radiopaque. The purpose of said pin(s) is to allow the platform to be affixed to the vertebral body or disc when the platform is gently pressed against that structure. In the preferred embodiment, the pins are serrated so that they may hold their position better and resist dislodgement once deployed.

On the upper and lower surface of the platform are notches which correspond to different distances (in millimeters) from the center pin. These notches serve as a place where marks can be made accurately on the surface of the spine with indelible ink or electrocautery. Also, within the body of the platform are circular perforations which also serve as additional points where surface marks may be made on the spine with indelible ink or electrocautery.

These and other objects are attained by a radiographic spine marker as described below.

### BRIEF DESCRIPTION OF THE DRAWINGS

In the accompanying drawings,

FIG. 1 is a front elevation of a radiographic spine marker embodying the invention;

FIG. 2 is a perspective view showing the marker from the rear; and

FIG. 3 is an x-ray image showing markings made on a marker applied to a patient.

### DESCRIPTION OF THE PREFERRED EMBODIMENT

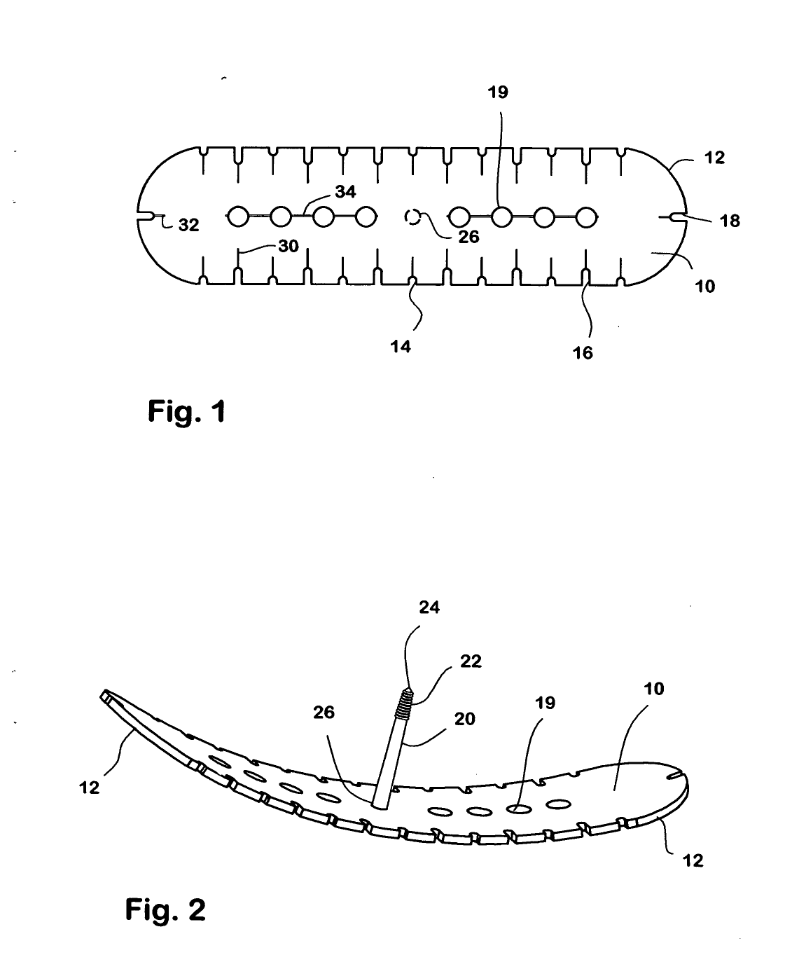

A radiographic spine marker embodying the invention, shown in FIG. 1, comprises a platform 10 having rounded ends 12. The platform's dimensions may be about 3.9 cm by 1.0 cm for an adult, but the dimensions can be changed according to the intended patient and the needs of the surgeon. The platform is radiolucent, and has a curvature designed to match that of the anterior portion of an average spinal vertebra or intervertebral disc. The platform is flexible so as to conform to minor variations in the curvature between individual spines. Various plastics, fabrics or synthetics may be employed to form the platform, provided they are radiolucent.

The front face of the marker platform has small and large notches 14, 16 along both its straight edges, and a notch 18 at either end as well. These notches serve as a place where marks can be made accurately on the surface of the spine. Within the body of the platform are circular perforations 19 which also serve as additional points where surface marks may be made on the spine. The marks can be made accurately on the surface of the spine with indelible ink or electrocautery.

Radiopaque hash marks 30 extend inward from each notch. A radiopaque centerline 34 of the platform is marked as well. The hash marks are separated by distances of one or more millimeters, depending on the accuracy needed for the surgical procedure in question. The marks are of sufficient size so as to be visible on a standard AP or fluoroscopic x-ray.

At least one post 20 is attached to the rear face of the marker, as shown in FIG. 2. The purpose of the post(s) is to allow the platform to be affixed to the vertebral body or disc when the platform is gently pressed against that structure. The post has a serrated tip 22 terminating at a point 24. The serrations allow the post to hold the platform in position better and resist dislodgement once deployed. The base 26 of the post is connected at the center of the rear surface of the platform by adhesives, welding, or other suitable means.

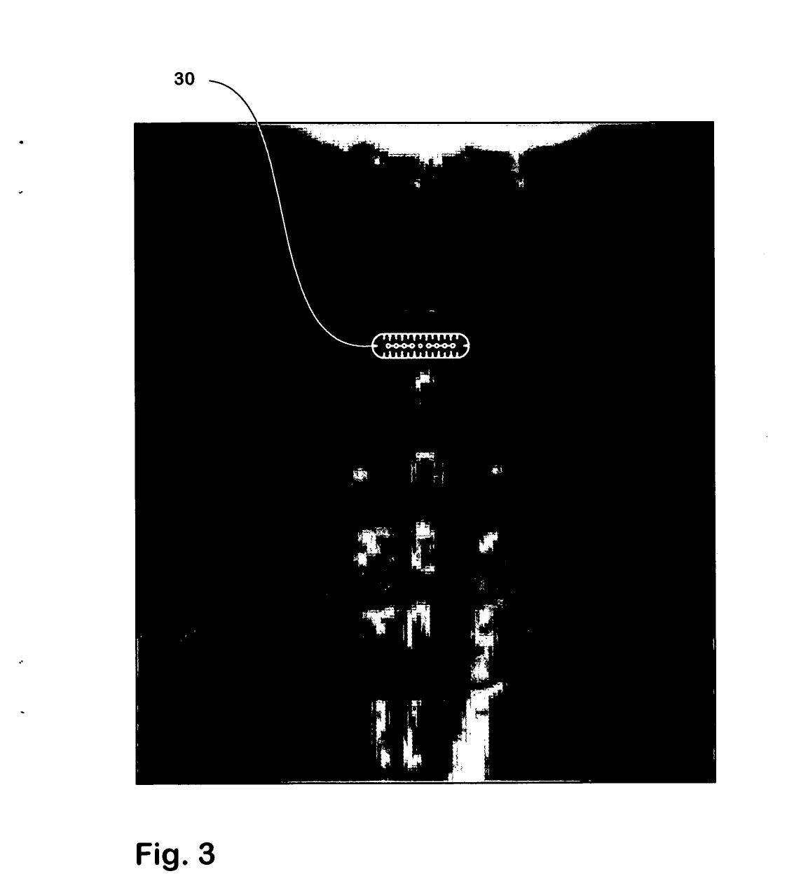

In use, the surgeon applies the platform over the spine. FIG. 3 shows an X-ray image of the device on the spine. The surgeon uses the platform to make accurate markings with indelible ink, identifying the location of the center of the spine. Once the marks have been made, the platform can be removed and discarded.

Since the invention is subject to modifications and variations, it is intended that the foregoing description and the accompanying drawings shall be interpreted as only illustrative of the invention defined by the following claims.

I claim:

1. A radiographic spine marker comprising a flexible, radiolucent platform having radiopaque markings thereon to act as a guide for a surgeon marking the location of the spine.

2. The invention of claim 1, wherein the markings are made on a front face of the platform, and further comprising at least one post extending from a rear face of the platform, for securing the platform to the patient.

3. The invention of claim 2, wherein at least one post has a pointed tip.

4. The invention of claim 2, wherein the post has serrations to resist dislodgement of the marker.

5. The invention of claim 1, wherein the markings include markings disposed at equal known intervals so that the marker can be used to measure spinal elements.

6. The invention of claim 1, further comprising a plurality of notches disposed along at least one edge of the platform, said notches being spaced at equal intervals.

7. The invention of claim 6, wherein the notches are formed along opposite edges of the platform.

8. The invention of claim 6, wherein at least some of the notches have different depths.

9. The invention of claim 8, wherein the notch depth alternates along the edge of the platform.

10. The invention of claim 6, wherein at least some of the radiopaque markings are disposed adjacent said notches, to indicate the position of the notches.

11. The invention of claim 1, further comprising a plurality of perforations formed in the platform through which marks may be made on the spine.
